(19) United States
(12) Patent Application Publication
(10) Pub. No.: US 2002/0020412 A1
(43) Pub. Date: Feb. 21, 2002

(54) Jet nebulizer assembly for home administration of drugs in aerosols

(76) Inventors: Brian E. Gilbert, Houston, TX (US); J. Vernon Knight, Houston, TX (US); Claire F. Verschraegen, Houston, TX (US)

Correspondence Address: Benjamin Aaron Adler, ADLER & ASSOCIATES, 8011 Candle Lane, Houston, TX, 77071, US

(21) Appl. No.: 09/919,446

(22) Filed: Jul. 31, 2001

Related U.S. Application Data
(60) Provisional application No. 60/222,406, filed on Aug. 2, 2000.

(51) Int. Cl. A61M 11/00; A61M 15/00; A61M 16/10

(52) U.S. Cl. 128/203.12; 128/200.21

(57) ABSTRACT

The present invention is directed to a jet nebulizer assembly for administering drugs via aerosols in a patient's home and a method of treating a cancer in a patient's home by utilizing the jet nebulizer assembly.

### CROSS-REFERENCE TO RELATED APPLICATIONS

This non-provisional application claims benefit of priority of provisional U.S. Ser. No. 60/222,406, filed Aug. 2, 2000, now abandoned.

### BACKGROUND OF THE INVENTION

1. Field of the Invention

The present invention relates generally to the fields of pharmacology and cancer treatment. More specifically, the present invention relates to a jet nebulizer assembly used for administering anti-cancer drugs in aerosols in patients' homes.

2. Description of the Related Art

In the lung, many different diseases have been treated successfully through utilization of aerosol delivery systems used t o deposit drugs directly on to the pulmonary surfaces. For delivery in this manner, a variety of devices have been developed, e.g., metered dose inhalers and dry powdered inhalers. Jet-nebulizers have been used clinically for aerosol delivery of water-soluble drugs and micronized suspensions; however, their use with water insoluble, hydrophobic compounds has been limited.

The development of liposomal and polymer formulations compatible with aerosol delivery has allowed the jet nebulizer to deliver additional drugs. Utilization of liposomes for aerosol delivery has many advantages, including aqueous compatibility and sustained pulmonary release allowing maintenance therapeutic drug levels. In addition, liposomes facilitate intra-cellular delivery, particularly to alveolar macrophages. Other vehicles for delivery of aerosols such as polyethylenimine (PEI) for genes may be used with this methodology.

The efficacy of localized, topical therapy via aerosols is determined by the amount of drug delivered at the sites of disease within the lungs. There are several different key parameters that determine the amount of delivery and thus, the therapeutic efficacy of aerosol formulations. For example, nebulizer design and variation, operating conditions (e.g., flow rate), and the presence of ancillary equipment (tubing, connectors, mouth pieces, face masks, and the like), are important variables. Thus, aerosol output efficiency can b e increased through proper implementation of the proper nebulizer device. Inappropriate implementation of the device and/or imperfect parameters can affect inhaled dosages, delivery sites and influence the therapeutic outcome.

The prior art is deficient in the lack of a nebulizer assembly and a method that could be used for administering drug aerosols in patients' homes. The present invention fulfills this long-standing need and desire in the art.

### SUMMARY OF THE INVENTION

In one embodiment of the present invention, there is provided a jet nebulizer assembly for administering drugs via aerosols in a patient's home. This jet nebulizer assembly comprises: a nebulizer having a top and a bottom end, wherein the bottom end of the nebulizer is connected to an air source; a first connector having at least two ends, wherein first end of the first connector is connected to the top end of the nebulizer; two tubing pieces, wherein first end of first tubing piece is connected to second end of the first connector; a second connector having three ends, wherein first end is connected to second end of the first tubing piece and second end is connected to second tubing piece; a face mask, to which third end of the second connector is connected; and a filter, which connects to the face mask via the second tubing piece.

In another embodiment of the present invention, there is provided a method of treating a cancer in a patient's home by delivering anti-cancer drugs in aerosols via the jet nebulizer assembly disclosed herein to the patient in need of such treatment.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the Aerotech II nebulizer, wherein the top end is connected to a “Y” assembly, the bottom end is connected to an air source.

FIG. 2 is a schematic drawing of the nebulizer assembly with Corr-A-Tubing and Mouth-only Face Mask.

FIG. 3 shows pharmacokinetics of liposomal 9-nitrocamptothecin (9-NC), wherein mean (+SD) plasma levels in 5 cancer patients are shown following treatment with 9-NC liposome aerosol by mouth-only breathing.

### DETAILED DESCRIPTION OF THE INVENTION

Topoisomerase-I inhibitors have the capability to eradicate human tumors in xenograft models. Therefore, human cancer cells are extremely sensitive to camptothecin. However camptothecin analogs are not curative in the clinical settings probably because of poor distribution of the camptothecin lactone to the tumor cells growing in humans. It was hypothesized that a modification of the formulation and a systemic delivery that avoids first pass in the liver may increase the therapeutic index. Aerosol delivery of liposomal 9-nitrocamptothecin may possibly delay opening of the lactone ring, through liposomation.

The present study demonstrates that delivery through aerosolization of fine particles is associated with systemic absorption, and perhaps with sustained levels of closed ring 9-nitrocamptothecin. Animal data (nude mice) shows minimal toxicity and no weight loss, with substantial antitumor activity at reduced doses against breast, lung, and colon cancer xenografts. The feasibility and safety o f administering 9-nitrocamptothecin is determined by aerosolization for 5 consecutive days per week.

The present invention is directed to a jet nebulizer assembly for administering drug aerosols in a patient's home. This jet nebulizer assembly comprises a nebulizer having a top and a bottom end, wherein the bottom end of the nebulizer is connected to an air source; a first connector having at least two ends, wherein first end of the first connector is connected to the top end of the nebulizer; two tubing pieces, wherein first end of first tubing piece is connected to second end of the first connector; a second connector having three ends, wherein first end is connected to second end of the first tubing piece and second end is connected to second tubing piece; a face mask, to which third end of the second connector is connected; and a filter, which connects to the face mask via the second tubing piece.

In a presently preferred embodiment of the assembly, the jet nebulizer produces aqueous aerosol particles having mass median aerodynamic diameter (MMAD) of from about 1 micron to about 3 microns, and the air source provides a flow rate of at least 10 L/min. Preferably, the air source is attached to a condensing system to remove water from the patient's room air so that sufficiently dry air with reduced humidity can be produced. Furthermore, the connector used to connect the tubing piece to the nebulizer can be in any shape, such as “Y”, “T”, “I”, or “L”, as long as the connector does not restrict o r reduce the air flow or aerosol content of the drugs. An example of the filter is a HEPA filter used to prevent exhaled drugs from releasing into surrounding environment. Examples of representative drugs which can be used in this jet nebulizer assembly include 9-nitrocamptothecin, 20S-camptothecin, 9-amino-camptothecin, 10,11-methylenedioxycamptothecin, taxol, taxol-A, mitotane, methotrexate, mercaptopurine, lomustine, interferon, 5-fluorouracil etopiside, p53 and Rb. These drugs may be carried in a vehicle such as water, liposomes, polymers, emulsions, micelles, nanoparticles or polyethylenimine (PEI).

The present invention is also directed to a method of treating a cancer in a patient's home by delivering drugs in aerosols via the jet nebulizer assembly of the present invention to the patient in need of such treatment. A specific example of the anti-cancer drug is 9-nitrocamptothecin. Preferably, the drugs are carried in a vehicle such as water, liposomes, polymers, emulsions, micelles, nanoparticles or polyethylenimine (PEI), and delivered at a dosage range of from about 1 μg/kg per day to about 100 μg/kg per day for 5 consecutive days per week for 8 weeks. During the treatment, the produced aerosol particles have mass median aerodynamic diameter (MMAD) of from about 1 micron to about 3 microns and are delivered under an air flow rate of at least 10 L/min. Examples of a disease, such as cancer, suitable for such treatment include a breast cancer, a lung cancer, a colon cancer, a cervix cancer, a leiomyosarcoma, an endometrial carcinoma, and a melanoma.

The following examples are given for the purpose of illustrating various embodiments of the invention and are not meant to limit the present invention in any fashion.

### EXAMPLE 1

Assembly of a Jet Nebulizer

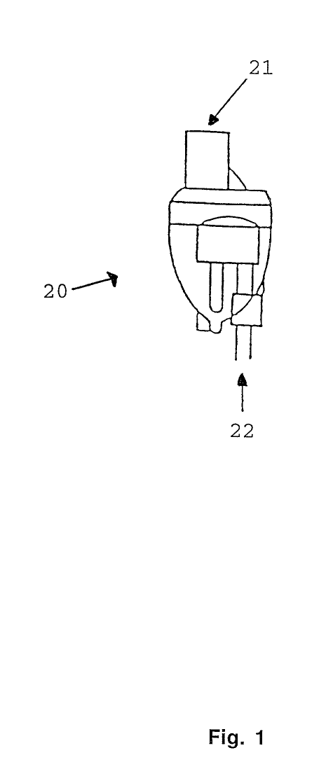

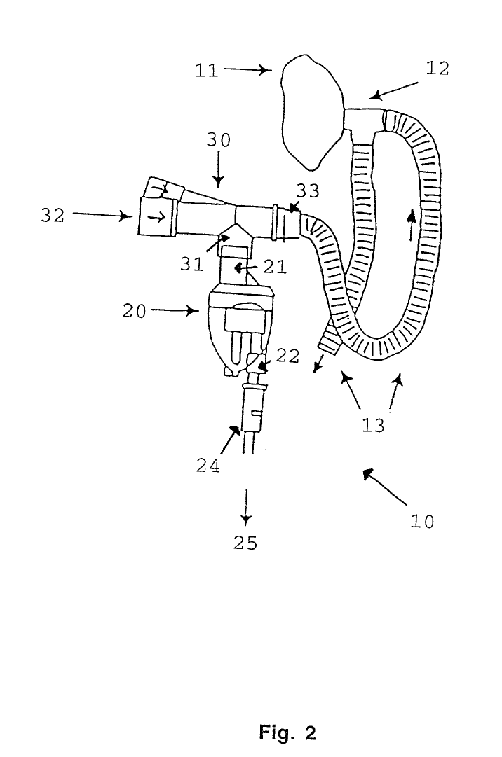

A jet nebulizer assembly 10, having a jet nebulizer 20, e.g. an Aerotech II nebulizer, is assembled (see FIGS. 1 and 2) according to the following steps: first, with the modified “Y” assembly 30 in a horizontal position, connect the nebulizer 20 to the open port of the “Y” 31 at the top end 21 and press firmly. Secondly, firmly attach the air vent end of the air tubing 24 to the bottom tip 22 of the nebulizer. Thirdly, attach the opposite end of the air tubing 24 to the air/O₂ supply 25 (compressor or tank, not shown). Pressure is set at 50 psi and the flow rate at 10 L/min. Fourthly, attach “Corr-A-Tubing” 13 to the “tail of the Y connecting piece” 33, located horizontally opposite to the one-way air valves 32, and the other end of the tubing 13 to a “T” connector 12 with a second piece of “Corr-A-Tubing” 13 hanging down from tail of the “T” connector 12. The “T” connector 12 is inserted into the front of a “mouth-only” face mask 11.

Following the assembly, the air compressor is plugged into an electrical outlet. The flow meter knob is turned all the way counter-clockwise to the “off” position

### EXAMPLE 2

Reconstituting 9-Nitrocamptothecin

9-Nitrocamptothecin is reconstituted freshly each time before use. For reconstituting, a 10 ml syringe with an 18-guage needle is used to add 10 ml of sterile, pyrogen-free water into a vial of powdered 9-Nitrocamptothecin (supplied by SP Pharmaceuticals, Albuquerque, N.M.). The vial is then shaken vigorously back and forth for 5 times.

### EXAMPLE 3

Filling Nebulizer with Drug

A 10 ml syringe with an 18-guage needle is used to remove the entire drug from the vial. The “Y” connector 31 is removed from the nebulizer assembly, and then the 10 ml reconstituted drug is added to the nebulizer 20 by emptying the syringe through the top end 21 of the nebulizer (see FIG. 1). The syringe was squeezed with a constant force. After emptying the syringe, the “Y” connector 31 is placed back on top end 21 of the nebulizer 20.

After adding drug to the nebulizer, 18-guage needle is reinserted into the used water vial and twisted off the syringe. The needle is then left in the vial and discarded in a safe container.

### EXAMPLE 4

Usage of the Nebulizer for Aerosol Treatment

The air tubing 24 must be connected from the compressor (not shown) to the nebulizer 20 (see FIG. 2). The “mouth-only” face mask 11 which is connected by “Corr-A-Tubing” 13 to the nebulizer 20 is then put on patients' face and secured firmly in place with the head cap and straps (not shown). The end of the “Corr-A-Tubing” 13 that is hanging down from the “T” connector 12 is hooked horizontally to the HEPA filter (not shown) with tape, string or a clip and secured in two places. The “on-off” switch is turned to the “ON” position. With the compressor 25 on, the flow rate is set to 10 L/min.

If additional treatments are needed, the nebulizer can be refilled. To refill the nebulizer, the compressor 25 “on-off” switch is turned to “OFF” position, and the air tubing 24 is disconnected from the bottom of the nebulizer 22. The “Y” connector 31 is removed from the top of the nebulizer 23. 10 ml of freshly reconstituted drug is added through the top of the nebulizer 20 with the syringe with an 18-guage needle as described above. Afterwards, the air tubing 24 is reconnected to the nebulizer 20, and the “on-off” switch is turned to the “ON” position. The air flow is ensured to be at 10 L/min. If not, the knob is turned to the correct flow.

When final treatment is completed, the compressor 25 is turned off with the “on-off” switch, and the “T” connector 12 and “Corr-A-Tubing” 13 are disconnected from the face mask 11. The HEPA filter (not shown) stays running for additional 5 minutes to remove any residual drug, after which the face mask 11 is removed.

Currently patients are treated for one hour a day, which requires 2 vials of drug. This treatment is given 5 days a week for 8 weeks.

### EXAMPLE 5

Removal of Exhaled Drug Using a Filtering System

After the aerosol treatment, the exhaled drug needs to be removed from the environment to prevent exposure to other individuals in the proximity. A HEPA filter system is used for this purpose. In the hospital a system called DeMistifier made by Peace Medical is generally used. However, DeMistifier is too big for home usage, instead, EnviroCare HEPA filter made by Honeywell is preferred. The idea is to attach the exhale tube that comes from the bottom of the face mask to the filter. By doing so, the exhaled drug is removed from the environment. An equivalent filtering system other than HEPA may be used.

### EXAMPLE 6

Cleaning of the Nebulizer and Connectors

Nebulizer can be reused. For cleaning, the nebulizer is washed well with warm water. A small amount of water is added and the nebulizer is connected to the air supply for 1-2 minutes. Water is then discarded and a small amount of 70% ethanol or isopropyl alcohol is added. The nebulizer is reconnected to the air supply for 1-2 minutes. Afterwards, the alcohol is discarded and the nebulizer is rinsed well with warm water. A small amount of water is added one again and the nebulizer is connected the to the air supply for 1-2 minutes. The water is again discarded and the nebulizer is reconnected to the air supply for 1-2 minutes to air dry. The dried nebulizer is ready for reuse. Each nebulizer may be reused for maximum 10 times.

To clean aerosol tubing and connecting piece(s), thoroughly rinse the tubing and connecting piece(s) with warm water at the end of each week, and let them air dry over the weekend. Afterwards, the tubing and connecting piece(s) are ready for reuse.

### EXAMPLE 7

Maintenance of Air Compressor and HEPA Filtering Unit

The compressor requires little maintenance. For once a week, the air filters are removed at the back of the compressor, rinsed in water and air-dried. The dried filters are put back in the compressor. If liquid condenses in the glass trap on the coil unit, one may press the button at the bottom while the pump is running. By doing so, the water will blow out of the bottom.

For HEPA filtering unit, one may follow the maintenance instructions supplied by the manufacturer. At times specified by the manufacturer, the charcoal and HEPA filters need to be replaced.

### EXAMPLE 8

Clinical Study

Patients with primary or metastatic disease to the lungs are enrolled in this phase I study if they fulfil the following eligibility criteria: pathologic diagnosis of cancer, failure after standard cancer treatment, performance status (Zubrod PS)<3, pulmonary function> 50% by spirometry and DLCO, normal organ functions, no symptomatic brain metastasis.

Treatment consisted of 6.7 μg/kg/day by aerosolization with a flow of 10 L/min. of air. In the feasibility cohort, treatment was given every day for 5 consecutive days, and repeated every 3 weeks if disease remained stable. Plasma was obtained on day 4 or 5 of therapy to determine the pharmacokinetic profile of the drug. Bronchoalveolar lavages to measure the amount of 9-NC were performed on consenting patients. Disease was evaluated by CT-scan of the chest every 2 courses.

### EXAMPLE 9

Clinical Study Results

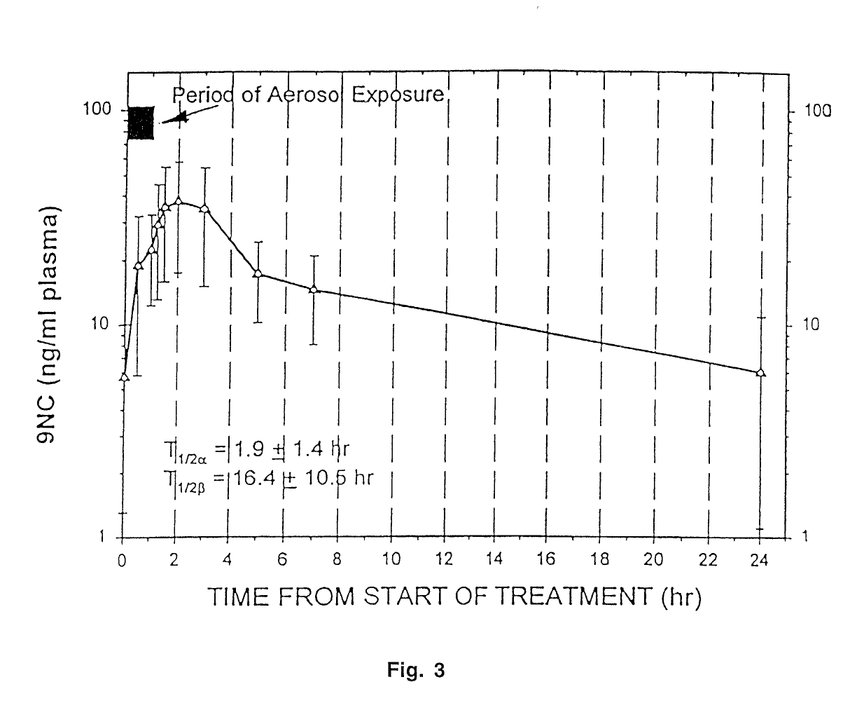

Six patients (4 woman, 2 men) were treated in the feasibility cohort. Patient characteristics included a median age of 57 years (3972 years), and a Zubrod PS of 0 (range 0-1). The median pulmonary function (FEV1/FVC) was 94% of control. All patients had received a median number of prior treatments of 2. Disease sites were cervix cancer (2 patients), leiomyosarcoma, endometrial carcinoma, lung cancer, and melanoma. Two to fourteen courses have been administered per patient. No grade ≧2 side effects have been observed at this low dose. 9-NC is absorbed systemically as determined by HPLC and mass spectrometry on the plasma (FIG. 3).

FIG. 3 also shows that maximum drug concentration is seen at 2 hours after the end of the aerosolization, with a mean concentration of 36.7 ng/ml (4 patients), falling to 4.9 ng/ml 24 hours later. Lactone was detected (<5 ng/ml) but decreased immediately after aerosolization. Stabilization of disease was observed in 2 patients. The study will accrue patients at higher doses and longer period of delivery.

Any patents or publications mentioned in this specification are indicative of the levels of those skilled in the art to which the invention pertains. These patents and publications are herein incorporated by reference to the same extent as if each individual publication was specifically and individually indicated to b e incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A jet nebulizer assembly for administering drugs via aerosols in a patient's home, wherein said nebulizer assembly comprises:
a jet nebulizer, wherein said nebulizer has a top and a bottom end, and wherein said bottom end of said nebulizer is connected to an air source;
a first connector having at least two ends, wherein said first end of said first connector is connected to said top end of said nebulizer;
two tubing pieces, wherein a first end of said first tubing piece is connected to said second end of said first connector;
a second connector having three ends, wherein said first end of said second connector is connected to said second end of said first tubing piece, and wherein said second end of said second connector is connected to a first end of said second tubing piece;
a face mask, wherein said face mask is connected to said third end of said second connector; and
a filter, wherein said filter is connected to said face mask via said second tubing piece.

2. The jet nebulizer assembly of claim 1, wherein said jet nebulizer produces aqueous aerosol particles having mass median aerodynamic diameter (MMAD) of from about 1 micron to about 3 microns.

3. The jet nebulizer assembly of claim 1, wherein said air source provides an air flow rate of at least 10 L/min.

4. The jet nebulizer assembly of claim 1, wherein said air source is attached to a condensing system to remove water from the patient's room air.

5. The jet nebulizer assembly of claim 1, wherein said first connector has a feature of maintaining air flow and aerosol content of the drugs.

6. The jet nebulizer assembly of claim 1, wherein said filter prevents exhaled drugs from releasing into surrounding environment.

7. The jet nebulizer assembly of claim 6, wherein said filter is a HEPA filter.

8. The jet nebulizer assembly of claim 1, wherein said drug is an anti-cancer drug selected from the group consisting of 9-nitrocamptothecin, 20-S-camptothecin, 9-amino-camptothecin, 10,11-methylenedioxy-camptothecin, taxol, taxol-A, mitotane, methotrexate, mercaptopurine, lomustine, interferon, 5-fluorouracil, etopiside, p53 and Rb.

9. The jet nebulizer assembly of claim 8, wherein said drug is carried in a carrier selected from the group consisting of water, liposomes, polymers, emulsions, micelles, nanoparticles and polyethylenimine.

10. A method for treating a disease in a patient's home, comprising the step of:
delivering anti-cancer drugs in aerosols via the jet nebulizer assembly of claim 1 to said patient.

11. The method of claim 10, wherein the drug aerosol has a mass median aerodynamic diameter (MMAD) of from about 1 micron to about 3 microns.

12. The method of claim 10, wherein said drug is carried in a carrier selected from the group consisting of water, liposomes, polymers, emulsions, micelles, nanoparticles and polyethylenimine.

13. The method of claim 10, wherein drug is selected from the group consisting of 9-nitrocamptothecin, 20-S-camptothecin, 9-amino-camptothecin, 10,11-methylenedioxy-camptothecin, taxol, taxol-A, mitotane, methotrexate, mercaptopurine, lomustine, interferon, 5-fluorouracil, etopiside, p53 and Rb.

14. The method of claim 10, wherein said drugs are delivered at a dosage range of from about 1 μg/kg per day to about 100 μg/kg per day for 5 consecutive days per week for 8 weeks.

15. The method of claim 10, wherein said drugs are delivered under an air flow rate of at least 10 L/min.
